(19) United States
(12) Patent Application Publication
(10) Pub. No.: US 2024/0415708 A1
(43) Pub. Date: Dec. 19, 2024

(54) COLOR COORDINATED AND REINFORCED WINGS ON FEMININE HYGIENE PRODUCTS

(71) Applicant: Amy Toledo, Los Angeles, CA (US)

(72) Inventor: Amy Toledo, Los Angeles, CA (US)

(21) Appl. No.: 18/745,425

(22) Filed: Jun. 17, 2024

Related U.S. Application Data
(60) Provisional application No. 63/509,022, filed on Jun. 19, 2023.

(51) Int. Cl. A61F 13/84; A61F 13/47; A61F 13/56

(52) U.S. Cl. CPC A61F 13/84; A61F 13/4702; A61F 13/4704; A61F 13/5616; A61F 2013/8497

(57) ABSTRACT

The present invention is a system and method for decreasing visibility of feminine products through clothing, wherein the non-absorbent material including the wings on the product, are made using colors that batter match clothing and may be used to attach the clothing protection product to undergarments of the user, the wings may be made stronger and resistant to tearing, adhesives used to attached the wings to an undergarment may be modified to allow easier detachment from the undergarment, and the bond between the wings and the non-absorbent layer may be reinforced to prevent the wings from tearing off the clothing protection product if the product must be detached and moved.

### BACKGROUND

Field of the Invention: This invention relates generally to absorbent clothing protection products. More specifically, the product is an absorbent menstrual pad that has wings for attachment to an undergarment, wherein the wings are not a part of but are attached to the absorbent material, and wherein the wings come in a variety of different colors so that menstrual pad is not as visible through clothing.

Description of Related Art: A variety of clothing protection products are sold on the open market. Some of these clothing protection products include patterns and others are offered in black and white. The apparent function of the patterns or the black and white colors are to provide some sort of adaptability for different colored or patterned clothing, with the ultimate goal being the ability to make the clothing protection product less visible through clothing.

For example, one particular product made a great effort to try and make certain that the color of the absorbent materials matched the color of the non-absorbent materials. This was difficult to do because the absorbent material often changed color when it was wet.

Accordingly, it would be an advantage over the prior art to simplify the color coordination of wings on a clothing protection product with clothing of a user. It would be another advantage to avoid any modifications to the absorbent material and use standard materials and colors for the absorbent material and offer a larger variety of colors in the non-absorbent material.

Another issue with the prior art is that the material used as the wings is not thicker than the non-absorbent layer that it is part of. This may become an issue when a user attaches the wings to an undergarment but then has to reposition the wings because the clothing protection product is not positioned correctly. The user must then pull the wings off the undergarment. The wings may be thin and flimsy and may then tear.

Accordingly, it would be an advantage over the prior art to make up to three additional improvements to the clothing protection product. First, the wings may be altered to thereby make them stronger and more resistant to tearing. Second, the adhesive used to attach the wings to the undergarment may be modified relative to present-day adhesives being used in clothing protection products. In this way, less force may need be applied when removing the wings from the undergarment for repositioning. Third, it may be possible to reinforce the wings and the non-absorbent layer to prevent the wings from tearing from the non-absorbent layer if the wings need to be moved.

### BRIEF SUMMARY

The present invention is a system and method for decreasing visibility of feminine products through clothing, wherein the non-absorbent material including the wings on the product, are made using colors that batter match clothing and may be used to attach the clothing protection product to undergarments of the user, the wings may be made stronger and resistant to tearing, adhesives used to attached the wings to an undergarment may be modified to allow easier detachment from the undergarment, and the bond between the wings and the non-absorbent layer may be reinforced to prevent the wings from tearing off the clothing protection product if the product must be detached and moved.

In a first aspect of the invention, only the non-absorbent material that includes the wings used to attach the product to undergarments would be made with a variety of different colors and/or patterns to better color coordinate with outer clothing of a user.

In a second aspect of the invention, the absorbent materials would not be modified in any way and would retain the existing colors and materials that are presently being used.

In a third aspect of the invention, the wings are modified to be more tear resistant by increasing the thickness of the wings.

In a fourth aspect of the invention, the adhesive may be modified to be low-tack, repositionable, or a reusable adhesive material.

In a fifth aspect of the invention, the wings and the non-absorbent layer may be reinforced to further prevent separation of the wings from the non-absorbent layer.

In a sixth aspect of the invention, the reinforcement around the outer edge of the non-absorbent layer and the wings would also be color coordinated to thereby decrease visibility of the clothing protection product through user clothing.

These and other embodiments of the present invention will become apparent to those skilled in the art from a consideration of the following detailed description taken in combination with the accompanying drawings.

### BRIEF DESCRIPTION OF THE SEVERAL VIEWS OF THE DRAWINGS

FIG. 1 is a top view of a menstrual pad that is manufactured in accordance with the principles of the first embodiment of the invention.

FIG. 2 is a bottom view of the menstrual pad shown in FIG. 1.

FIG. 3 is a cut-away profile view of the layers in the first embodiment.

FIG. 4 is a bottom view of a second embodiment of the menstrual pad that includes stitching.

FIG. 5 is an alternate shape for a menstrual pad.

FIG. 6 is a bottom view of the menstrual pad that includes decorative elements on the wings.

### DETAILED DESCRIPTION

Reference will now be made to the drawings in which the various embodiments of the present invention will be discussed so as to enable one skilled in the art to make and use the invention. It is to be understood that the following description illustrates embodiments of the present invention and should not be viewed as narrowing the claims which follow.

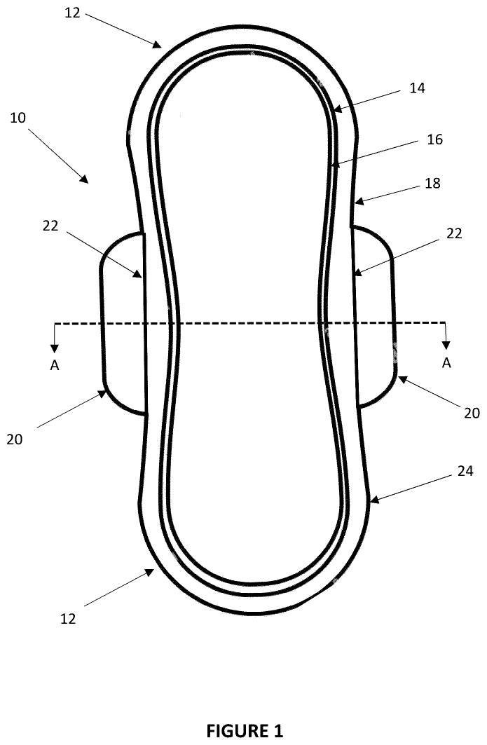

FIG. 1 is a top view of a first embodiment of a personal hygiene product that has been modified in accordance with the principles of the invention. In this first embodiment, the personal hygiene product is a menstrual pad 10. The top view shows a skin-facing side of the menstrual pad 10 as opposed to a bottom or opposite side of the menstrual pad that only shows an outer surface thereof.

In this embodiment, the menstrual pad 10 may have a substantially rectangular shape that is generally going to be flat or planar when not being used. The rectangular shape may have rounded ends 12 and may be comprised of a plurality of different layers. The exact shape of the rounded ends 12 may vary without departing from the scope of the first embodiment. The substantially rectangular shape may also become narrower in the middle so that the menstrual pad 10 is easier to wear, but this is not a requirement of the first embodiment. Therefore, the menstrual pad 10 may not become narrower in the middle and still be within the scope of the first embodiment.

FIG. 1 shows an outline of different layers of the menstrual pad 10. The menstrual pad 10 may include a first layer 14 that is in contact with skin, an absorbent second layer 16, and a non-absorbent third layer 18.

The first layer 14 may be an absorbent material that wicks moisture away from the skin. In this first embodiment, bodily fluids will pass through the first layer to the second layer 16 underneath.

The second layer 16 may be comprised of one or more absorbent materials that absorb the bodily fluids that pass through the first layer 14. It should be understood that the first layer 14 may completely cover the second layer 16 so that the bodily fluids that are absorbed are kept away from direct contact with skin.

The third layer 18 may be waterproof so as to contain the bodily fluids absorbed by the second layer 16. The third layer 18 may function as a buffer that provides protection to clothing, such as an undergarment, from the bodily fluids. In this first embodiment, the third layer is a non-white color. The third layer is a non-white color that is selected by the user to match the color of the undergarments being worn by the user and is referred to as color matching.

It should also be understood that while the first embodiment is directed to the third layer 18 having a non-white color so that it may match a color of an undergarment such as panties, in a second embodiment, the third layer may be white. While the prior art is also comprised of white menstrual pads 10, the prior art does not include the other modifications to the menstrual pad as defined by the first embodiment and which are also included in the second embodiment.

Another essential feature of the third layer 18 are the wings 20. The wings 20 are formed as extensions that are in the same plane as the menstrual pad 10 and that extend outwards from the narrower portion of the menstrual pad. The wings 20 are not attached as separate elements but are an integral part of the third layer 18. However, after the third layer 18 is formed, additional non-absorbent material may be added just to the wings 20. This additional non-absorbent material may be the same non-absorbent material of the third layer 18, or it may be a different non-absorbent material. What is important is that the additional non-absorbent material is added to the wings 20 to thereby increase the thickness and the strength of the wings. More than one layer of non-absorbent material may be added to the wings 20 to thereby increase the thickness.

There may also be more than one purpose for the additional thickness of the wings 20. For example, the additional thickness of the wings 20 may make it easier to fold the wings 20 when the menstrual pad 10 is attached to an undergarment. With the additional thickness, the wings 20 will naturally bend along the line 22 shown in FIG. 1. This line does not indicate a point of attachment but serves as a delineation between the thicker wings 20 and the thinner portion of the third layer 18. The line 22 may be straight or curved.

The additional thickness of the wings 20 may also prevent tearing of the wings if after attachment of the menstrual pad 10 to an undergarment, it is necessary to reposition the wings. The user will have to detach the wings 20 from the undergarment by pulling on the wings until an adhesive on the wings detaches from the undergarment. The thicker wings 20 may be less likely to tear.

The wings 20 of the first embodiment may include any structure on the menstrual pad 10 that is designed to enable the menstrual pad to be coupled to an undergarment. Accordingly, the exact shape of the wings 20 may vary greatly between different products that perform the same function and should not be considered a limitation of the claims.

It should also be understood that the outline of the menstrual pad 10 shown in FIG. 1 may vary between different brands. Furthermore, even a single brand may have more than one design that includes arcuate portions and linear portions or a combination of the two. What is important is that the design of the menstrual pad 10 is not a limitation of the present invention and thus all outlines and specific placement of absorbent and non-absorbent portions of the menstrual pads 10 should be considered to fall within the scope of the claims of the invention.

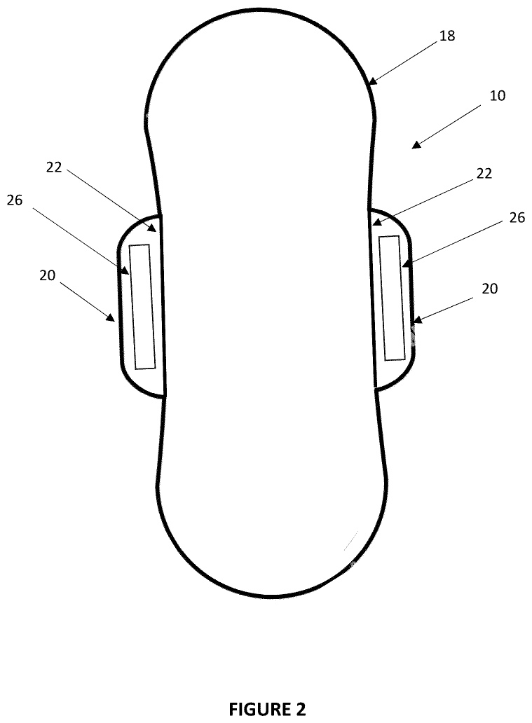

It is an important aspect of the invention that all of the visible elements of the menstrual pad 10 when it is attached to the undergarment that is being worn include the color matching feature of the third layer 18 to thereby minimize visibility of the menstrual pad through outer clothing. The visible elements of the third layer 18 are thus a bottom view that is not shown in FIG. 1 but is instead shown in FIG. 2.

FIG. 2 is a bottom view of the menstrual pad 10 shown in FIG. 1 and which only shows a reverse side of the third layer 18. The wings 20 are visible, and the lines 22 again show the delineation between the thicker wings 20 and the rest of the third layer 18.

Another feature of the first embodiment is an adhesive 26 that is disposed on the wings 20 and used to secure the menstrual pad 10 to the undergarment. The adhesive used in the prior art is strong enough that trying to remove the wings 20 from the undergarment once it has been attached is a difficult task which may result in tearing of the wings. The user is then forced to get another menstrual pad 10 and begin again.

Accordingly, it is another aspect of the first embodiment to reduce the tackiness of the adhesive 26 being used to secure the menstrual pad 10 to the undergarment. Even with the reinforced wings 20 of the first embodiment, removal of the wings in order to reposition them on the undergarment may also be easier if the adhesive 26 is not as strong as taught in the prior art. Thus, the adhesive on the wings may be a low-tack, repositionable, or a reusable adhesive material that enables the menstrual pad 10 to be repositionable on the undergarment.

It is important to understand that the only portion of the menstrual pad 10 that is changed in the first embodiment is the third layer 18, wherein the changes include a color of the third layer, a thickness of the wings 20, and the type of adhesive that is used on the wings to secure them to the menstrual pad. The method of manufacturing and the materials being used for the menstrual pad 10 would have no modifications other than the color and the adhesive 26 in the first embodiment.

The third layer 18 may or may not require modification in order to incorporate the color into the material. The original materials used for the third layer 18 may only need to be dyed the desired colors that would be offered by the manufacturer of the menstrual pads 10.

The color matching feature may be defined as making the third layer 18 of the menstrual pad 10 in any desired color. Thus, the color matching feature is providing the third layer 18 in any color that will limit the visibility of the menstrual pad 10 through undergarments and through clothing. Thus, if the undergarments are a shade of blue, the third layer 18 of the menstrual pad 10 may be a matching or a similar shade of blue.

While FIG. 1 shows the top view of the menstrual pad 10 that is disposed against the skin of the user, the bottom view of the menstrual pad 10 is comprised entirely of the reverse side of the third layer 18. The reverse side is thus facing outwards from the user. Furthermore, at least a portion of the reverse side of the menstrual pad 10 may be covered by the undergarments of the user.

The menstrual pad 10 is disposed on an inside surface of the undergarment and positioned at the bottom of the undergarment which is referred to as the gusset. The wings 20 are folded away from the top of the menstrual pad 10 and toward the reverse side. The wings 20 are then wrapped around and against an outer side of the gusset of the undergarment. In order to only show the color of the third layer 18, both sides of the wings 20 may need to be colored.

The color matching feature may be offered in any products that may be attached to the undergarments of the user, and not just those used for feminine hygiene products. If the product being used may be visible through or to the side of the undergarments, then the principles of the first embodiment are applicable.

The possible products that may take advantage of the color matching attributes of the first embodiment should be considered to include, but not be limited to, disposable diapers, pull-on diapers, training pants, sanitary napkins, pantiliners, incontinence briefs, and the like. Such products are those that are used for absorbing and containing urine and other bodily fluids. As such, these products and the present invention are not limited to females.

While the wings 20 of the menstrual pad 10 are highly visible because they wrap around the user's undergarment, any portion of the menstrual pad that might extend outside the edges of the undergarment could be visible through outer clothing of the user.

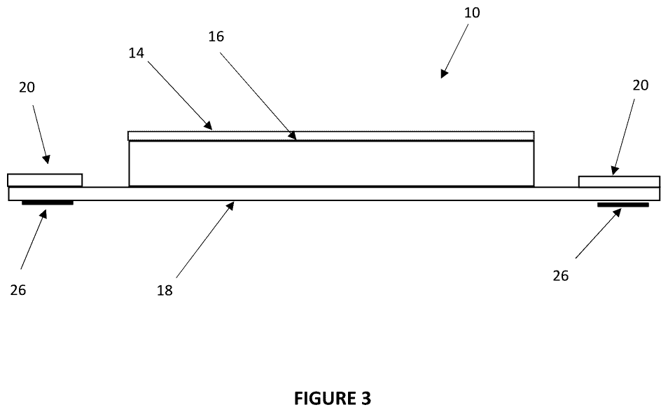

FIG. 3 is a cross-sectional view of the menstrual pad 10 as defined by line AA in FIG. 1. The menstrual pad 10 of the first embodiment may have the same plurality of layers that are found in all menstrual pads in the prior art, but with modifications. The thickness and dimensions of the layers shown in FIG. 2 may not be to scale and are only shown to illustrate the layers. The figure also does not show that the first layer 14 seals the absorbent second layer 16 inside.

In FIG. 3, the absorbent second layer 16 is sealed between the first layer 14 and the third layer 18. The thicker wings 20 are also shown as part of the third layer 18, with the adhesive 26 shown on the bottom side thereof.

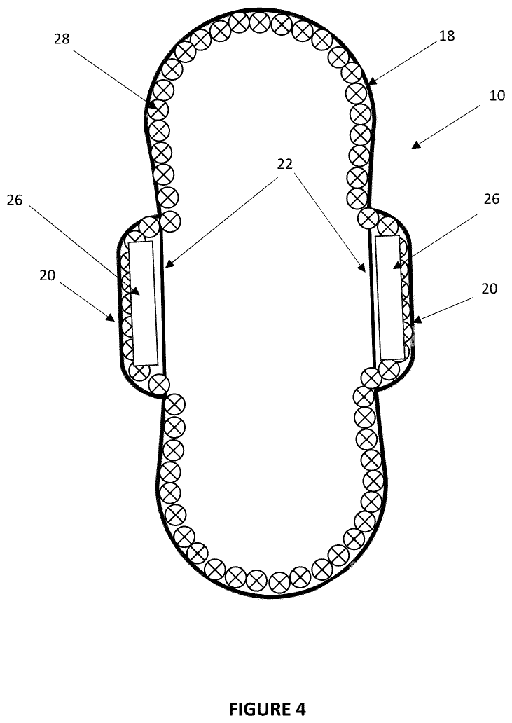

FIG. 4 is a top view of a second embodiment of the invention. The second embodiment includes all of the features of the first embodiment, namely, color matching of the third layer with the color of the undergarment, the use of thicker wings 20, and the use of a less tacky adhesive material on the wings that enable them to be more easily repositioned when being attached to the gusset of the undergarment.

A new aspect of the second embodiment is the addition of stitching 28 that reinforces and strengthens the outer edge 24 of the third layer 18. The thread used in any stitching 28 may also be an important material to color match with the third layer 18. The stitching 28 may also be used to reinforce the first layer 14 and the second layer 16.

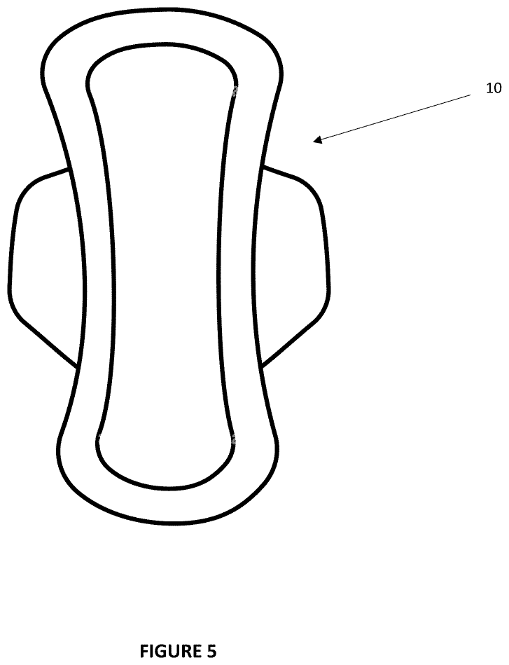

FIG. 5 is a top view of a menstrual pad 30 having a different shape for the perimeter of the pad. In this figure, the perimeter of the menstrual pad 30 has less of an arcuate shape and appears more rectangular and squared at the corners thereof.

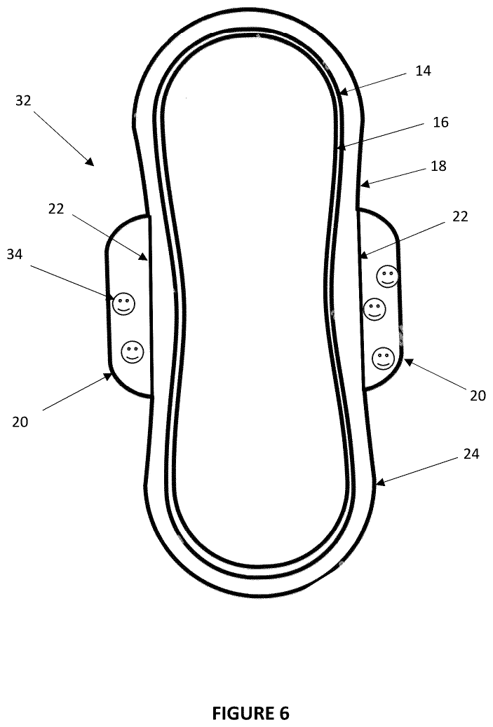

FIG. 6 is a top view of a menstrual pad 32, wherein the wings 20 may include decorative elements 34 that are attached to a side of the wings that face away from the undergarment when attached to the undergarment.

The wings 20 include decorative elements 34 such as beads, small images, or other decorations that may be applied to the wings 20 such that when they are folded and attached by the adhesive to the undergarment, the decorative elements on the wings are visible.

In summary, the invention is a personal hygiene product that is worn under or attached to an undergarment of a user and is color coordinated therewith, said personal hygiene product comprising a menstrual pad disposed in a plane and having a substantially rectangular shape that is contoured such that it narrows in a middle portion, wherein the menstrual pad is comprised of a first layer that is in contact with skin, an absorbent second layer, and a non-absorbent third layer, wherein the first layer is an absorbent material that wicks moisture away from the skin, wherein the absorbent second layer is disposed between the first layer and the third layer of the menstrual pad, wherein the absorbent second layer absorbs bodily fluids that pass through the first layer, wherein the third layer is waterproof and provides a buffer between the second layer and the undergarment, and wherein the third layer is a non-white color, two planar wings formed as extensions of the third layer, extending outwards from the menstrual pad, disposed in the plane thereof, and disposed on either side of the menstrual pad where it narrows, wherein the two wings are reinforced by being thicker than the third layer such that the two wings fold on a line defined by the location where the two wings extend outwards from the menstrual pad, and adhesive strips disposed on an underside of the two wings so that the wings may be attached to an outside surface of the undergarment to hold the menstrual pad in place.

Furthermore, the invention also comprises a method of using a personal hygiene product that is worn inside an undergarment of a user, and color coordinated therewith, said method comprising providing a menstrual pad disposed in a plane and having a rectangular shape that is contoured such that it narrows in a middle portion, wherein the menstrual pad is comprised of a first layer that is in contact with skin, an absorbent second layer, and a third layer, wherein the first layer is an absorbent material that wicks moisture away from the skin, wherein the absorbent second layer is disposed between the first layer and the third layer of the menstrual pad, wherein the absorbent second layer absorbs bodily fluids that pass through the first layer, wherein the third layer is waterproof and provides a buffer between the absorbent second layer and the undergarment, and wherein the third layer is a non-white color; providing planar wings formed as extensions of the third layer, extending outwards from the menstrual pad, disposed in the plane thereof, and disposed on either side of the menstrual pad where the menstrual pad narrows, wherein the two wings are reinforced by being thicker than the third layer such that the two wings fold on a line defined by the location where the two wings extend outwards from the body, and providing adhesive strips disposed on an underside of the two wings so that the wings may be attached to the undergarment to hold the menstrual pad in place, exposing the adhesive strips, disposing the menstrual pad on an inner surface and on a gusset of the undergarment with the first layer facing away from the gusset, and folding the two wings so that the gusset is at least partially disposed between the third layer and the two wings so that the adhesive strips are disposed against an outer surface of the undergarment.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. It is the express intention of the applicant not to invoke 35 U.S.C. § 112, paragraph 6 for any limitations of any of the claims herein, except for those in which the claim expressly uses the words ‘means for’ together with an associated function.

What is claimed is:

1. A personal hygiene product that is worn under or attached to an undergarment of a user and is color coordinated therewith, said personal hygiene product comprising;
a menstrual pad disposed in a plane and having a substantially rectangular shape that is contoured such that it narrows in a middle portion, wherein the menstrual pad is comprised of a first layer that is in contact with skin, an absorbent second layer, and a non-absorbent third layer, wherein the first layer is an absorbent material that wicks moisture away from the skin, wherein the absorbent second layer is disposed between the first layer and the third layer of the menstrual pad, wherein the absorbent second layer absorbs bodily fluids that pass through the first layer, wherein the third layer is waterproof and provides a buffer between the second layer and the undergarment, and wherein the third layer is a non-white color;
two planar wings formed as extensions of the third layer, extending outwards from the menstrual pad, disposed in the plane thereof, and disposed on either side of the menstrual pad where it narrows, wherein the two wings are reinforced by being thicker than the third layer such that the two wings fold on a line defined by the location where the two wings extend outwards from the menstrual pad; and
adhesive strips disposed on an underside of the two wings so that the wings may be attached to an outside surface of the undergarment to hold the menstrual pad in place.

2. The personal hygiene product as defined in claim 1 wherein the two wings are further comprised of at least one additional layer of material to increase the thickness of the two wings relative to the third layer; and
wherein the menstrual pad is disposed on an inner surface and on a gusset of the undergarment with the first layer facing away from the gusset and the third layer making contact with the gusset, and wherein the two wings are folded so that the gusset is at least partially disposed between the third layer and the two wings.

3. The personal hygiene product as defined in claim 2 wherein the third layer is further comprised of:
trimming disposed around an edge of the third layer, wherein the trimming adds structural integrity to the menstrual pad, and wherein the trimming is the same non-white color as the third layer; and
wherein the non-white color of the third layer matches the non-white color of the undergarment.

4. The personal hygiene product as defined in claim 3 wherein the adhesive strips utilize an adhesive material that is low-tack, repositionable, or a reusable adhesive material that enables the clothing protection product to be repositioned on the undergarment.

5. The personal hygiene product as defined in claim 1 wherein the two wings are further comprised of decorative elements that are attached to the two wings.

6. The personal hygiene product as defined in claim 1 wherein the non-white color is any color and includes lighter and darker shades of the non-white color.

7. The personal hygiene product as defined in claim 6 wherein the non-white color includes a plurality of different colors.

8. The personal hygiene product as defined in claim 1 wherein the non-white color includes a print or pattern that includes a plurality of different colors.

9. The personal hygiene product as defined in claim 1 wherein the personal hygiene product is selected from the group of personal hygiene products comprised of menstrual pads, disposable diapers, pull-on diapers, training pants, sanitary napkins, pantiliners, and incontinence briefs.

10. A method of using a personal hygiene product that is worn inside an undergarment of a user, and color coordinated therewith, said method comprising:
providing a menstrual pad disposed in a plane and having a rectangular shape that is contoured such that it narrows in a middle portion, wherein the menstrual pad is comprised of a first layer that is in contact with skin, an absorbent second layer, and a third layer, wherein the first layer is an absorbent material that wicks moisture away from the skin, wherein the absorbent second layer is disposed between the first layer and the third layer of the menstrual pad, wherein the absorbent second layer absorbs bodily fluids that pass through the first layer, wherein the third layer is waterproof and provides a buffer between the absorbent second layer and the undergarment, and wherein the third layer is a non-white color;
providing planar wings formed as extensions of the third layer, extending outwards from the menstrual pad, disposed in the plane thereof, and disposed on either side of the menstrual pad where the menstrual pad narrows, wherein the two wings are reinforced by being thicker than the third layer such that the two wings fold on a line defined by the location where the two wings extend outwards from the body; and
providing adhesive strips disposed on an underside of the two wings so that the wings may be attached to the undergarment to hold the menstrual pad in place;
exposing the adhesive strips;
disposing the menstrual pad on an inner surface and on a gusset of the undergarment with the first layer facing away from the gusset; and
folding the two wings so that the gusset is at least partially disposed between the third layer and the two wings so that the adhesive strips are disposed against an outer surface of the undergarment.

11. The method as defined in claim 10 wherein the method further comprises adding at least one additional layer of material to the two wings to thereby increase the thickness of the two wings relative to the third layer.

12. The method as defined in claim 11 wherein the method further comprises:
disposing trimming around an edge of the third layer, wherein the trimming adds structural integrity to the personal hygiene product, and wherein the trimming is the same non-white color as the third layer; and
matching the non-white color of the third layer to the non-white color of the undergarment.

13. The method as defined in claim 12 wherein the method further comprises utilizing an adhesive material for the adhesive strips that is low-tack, repositionable, or a reusable adhesive material so that the personal hygiene product is capable of being repositioned on the undergarment.

14. The method as defined in claim 10 wherein the method further comprises attaching decorative elements to the two wings.

15. The method as defined in claim 10 wherein the method further comprises making the non-white color to be any color including lighter and darker shades of the non-white color.

16. The method as defined in claim 15 wherein the method further comprises making the non-white color to be a plurality of different colors.

17. The method as defined in claim 16 wherein the method further comprises making the non-white color to be a print or pattern that includes a plurality of different colors.

18. The method as defined in claim 10 wherein the method further comprises selecting the personal hygiene products from the group of personal hygiene products comprised of menstrual pads, disposable diapers, pull-on diapers, training pants, sanitary napkins, pantiliners, and incontinence briefs.

19. A personal hygiene product that is worn under or attached to an undergarment of a user and is color coordinated therewith, said product comprising;
a menstrual pad having a generally planar and rectangular shape that is contoured such that it narrows in a middle portion, wherein the menstrual pad is comprised of a first layer that is in contact with skin, an absorbent second layer, and a third layer, wherein the first layer is a permeable material that wicks moisture away from the skin, wherein the absorbent second layer is disposed between the first layer and the third layer of the menstrual pad, wherein the absorbent second layer absorbs bodily fluids that pass through the first layer, and wherein the third layer is waterproof and provides a buffer between the absorbent second layer and the undergarment;
two wings formed as extensions of the third layer and disposed on either side of the menstrual pad where the menstrual pad narrows, wherein the two wings are reinforced by being thicker than the third layer of the menstrual pad such that the two wings fold on a line defined by where the two wings meet the menstrual pad; and
adhesive strips disposed on an underside of the two wings so that the wings may be attached to the undergarment to hold the menstrual pad in place.

20. The personal hygiene product as defined in claim 19 wherein the third layer and the two wings are further comprised of a non-white color.

21. The personal hygiene product as defined in claim 19 wherein the personal hygiene product is selected from the group of personal hygiene products comprised of disposable diapers, pull on diapers, training pants, sanitary napkins, pantiliners, and incontinence briefs.
